(19) United States
(12) Patent Application Publication
(10) Pub. No.: US 2021/0407078 A1
(43) Pub. Date: Dec. 30, 2021

(54) METHOD AND SYSTEMS FOR MEDICAL IMAGE PROCESSING USING A CONVOLUTIONAL NEURAL NETWORK (CNN)

(71) Applicant: PERIMETER MEDICAL IMAGING INC., Dallas, TX (US)

(72) Inventors: Navid SAMAVATI, Toronto, Ontario (CA); David REMPEL, Toronto, Ontario (CA)

(21) Appl. No.: 17/290,028

(22) PCT Filed: Oct. 29, 2019
(86) PCT No.: PCT/CA2019/051532

§ 371 Date: Apr. 29, 2021

Related U.S. Application Data
(60) Provisional application No. 62/752,735, filed on Oct. 30, 2018.

(51) Int. Cl. G06T 7/00; G06T 7/11; G06T 7/143; G06K 9/62; G16H 50/20; G16H 30/20; G16H 30/40; G06N 3/04

(52) U.S. Cl. CPC G06T 7/0012; G06T 7/11; G06T 7/143; G06K 9/6256; G06K 9/6265; G16H 50/20; G06T 2207/20016; G16H 30/40; G06N 3/04; G06T 2207/20076; G06T 2207/10101; G06T 2207/30024; G06T 2207/20084; G16H 30/20

(57) ABSTRACT

A system includes an imager configured to acquire at least one image of a tissue, a memory configured to store processor executable instructions, and a processor operably coupled to the imager and the processor. Upon execution of the processor executable instructions, the processor is configured to train a convolutional neural network (CNN) using a plurality of training images, and implement the CNN to determine a probability of abnormality of at least one region of tissue in the at least one image.

### CROSS-REFERENCE TO RELATED APPLICATIONS

This application claims priority to and the benefit of U.S. Provisional Patent Application No. 62/752,735, filed Oct. 30, 2018, titled “METHODS AND SYSTEMS FOR MEDICAL IMAGE PROCESSING USING A CONVOLUTIONAL NEURAL NETWORK (CNN),” the disclosure of which is incorporated by reference herein.

### BACKGROUND

Embodiments described herein relate to methods and systems for processing medical images, including optical coherence tomography (OCT) images. OCT is an imaging modality that provides three dimensional (3D), high resolution, cross sectional information of a sample. The information in a single OCT reflectivity profile contains information about the size and density of optical scatters, which can in turn be used to determine the type of tissue that is imaged.

One application of OCT is for the assessment of tumor margins in wide local excisions procedures, particularly breast conservation procedures. On average, about 25% of women who undergo breast conservation surgery require a second surgery, also referred to as re-excision, due to positive tumor margins, i.e., residual tumor has been left behind. Based on feedback from clinicians and a recently published study, presence of positive tumor margins or abnormal tissues at the margin has been shown to be the primary factor for re-excisions. One example of such abnormal tissue can be ductal carcinoma in situ (DCIS). OCT images of excised tissues can be employed to identify positive tumor margins in the operating room so as to decide whether additional excision should be performed right away, e.g., before suturing. However, there are no standards to interpret the OCT images and amount of raw data of these OCT images is usually very large. Accordingly it is challenging for physicians to make such decision within a short period of time and within the operating room based on the OCT images.

### SUMMARY

Embodiments described herein relate to a method and system for processing medical images. In some embodiments, the system includes an imager configured to acquire at least one image of a tissue, a memory configured to store processor executable instructions, and a processor operably coupled to the imager and the processor. Upon execution of the processor executable instructions, the processor is configured to train a convolutional neural network (CNN) using a plurality of training images, and implement the CNN to determine a probability of abnormality of at least one region of tissue in the at least one image. In some embodiments, the CNN can be implemented to identify at least one region of abnormal tissue (e.g., malignant tissue).

### BRIEF DESCRIPTION OF THE DRAWINGS

FIG. 1 is a schematic illustration of a convolutional neural network (CNN) for processing images, according to an embodiment.

FIG. 2 illustrates the decrease and increase of data size in the CNN shown in FIG. 1 via convolution and de-convolutions, respectively, according to an embodiment.

FIG. 3 is a schematic illustration of the system including a CNN to process medical images, according to an embodiment.

FIGS. 4A-4C illustrate a method of obtaining ground truth to train the CNN in the system shown in FIG. 3, according to an embodiment.

FIGS. 5A-5I show representative optical coherence tomography (OCT) B-scans and corresponding permanent section histology of ductal carcinoma in situ (DCIS) cases, according to an embodiments.

FIGS. 6A-6G show an example labeling of DCIS for training and using the system 300, according to an embodiments.

FIGS. 7A-7D illustrate a method of medical imaging processing using a tiered approach, according to an embodiment.

### DETAILED DESCRIPTION

Embodiments described herein relate to a method and system for processing medical images. In some embodiments, the system includes an imager (e.g., imaging device) configured to acquire at least one image of a tissue, a memory configured to store processor executable instructions, and a processor operably coupled to the imager and the processor. Upon execution of the processor executable instructions, the processor is configured to train a convolutional neural network (CNN) using a plurality of sample images (e.g., training images), and implement the CNN to identify at least one ductal carcinoma in situ (DCIS) in the at least one image.

Recent developments of OCT include expanding the field of view of a standard OCT system to facilitate the scanning of large sections of excised tissue in an operating room. This method of imaging generates large amounts of data for review, thereby posing additional challenges for real-time clinical decision making. Computer aided detection (CADe) techniques can be employed to automatically identify regions of interest, thereby allowing the clinician to interpret the data within the time limitations of the operating room.

In previous approaches of CADe, a classifier was trained using several features extracted from the OCT reflectance profile and then employed to differentiate malignant from benign tissue. More details about these approaches can be found in Mujat M, Ferguson R D, Hammer D X, Gittins C, Iftimia N. Automated algorithm for breast tissue differentiation in optical coherence tomography. J Biomed Opt. 2009; 14(3):034040; Savastru, Dan M., et al. “Detection of breast surgical margins with optical coherence tomography imaging: a concept evaluation study.” Journal of Biomedical optics 19.5 (2014): 056001; and Bhattacharjee, M., Ashok, P. C., Rao, K. D., Majumder, S. K., Verma, Y., & Gupta, P. K. (2011). Binary tissue classification studies on resected human breast tissues using optical coherence tomography images. Journal of Innovative Optical Health Sciences, 4(01), 59-66, each of which is herein incorporated by reference in its entirety.

Similar methods can also be employed to develop a classifier to identify exposed tumor. However, in a real clinical setting, large exposed tumor masses are not the primary driver for re-excisions. Instead, decisions to conduct additional excisions are mostly based on the presence (or absence) of small focal regions of carcinoma, for example ductal carcinoma in situ (DCIS).

A number of different attempts were made to identify DCIS in OCT data using standard image analysis with limited success. Segmentation (using region growing), edge, corner, and shape detection, and dictionary-based feature extraction using sparse coding all failed to produce any promising classifications for DCIS or benign duct detection.

To address above challenges, methods and systems described herein employ a convolutional neural network (CNN) to identify abnormal tissues (e.g., DCIS) in medical images. Since the beginning of the current decade, CNNs have shown great success in extracting features from various forms of input data. They have enabled unprecedented performance improvements in image classification, detection, localization, and segmentation tasks. CNNs work by hierarchically extracting features from input images in each layer, and performing classification at the last layer typically based on a linear classifier. By modifying the activation function of the neurons and providing a substantial amount of input training data, one can effectively find the optimal weights of such networks, and classify virtually any region of interest with impressive accuracy.

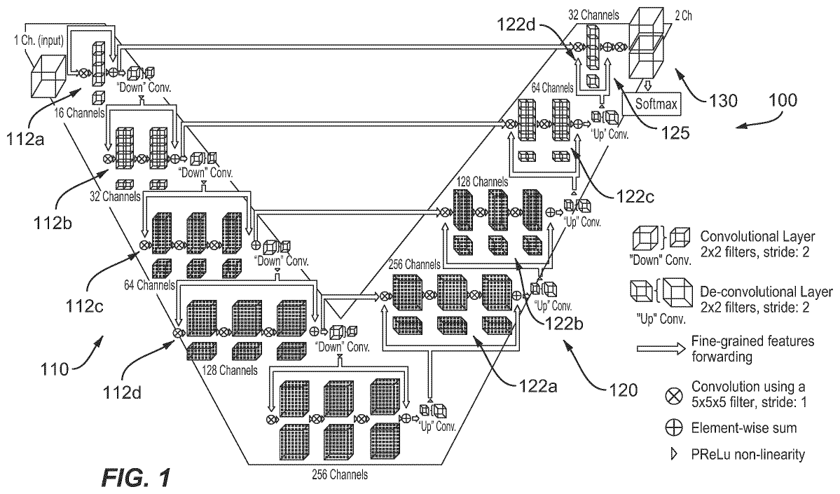

FIG. 1 shows a schematic of a CNN 100 used for identifying abnormal tissues (e.g., DCIS) in medical images. In some embodiments, the CNN 100 can be used to determine a probability of abnormality, as further detailed below. The CNN 100 has a symmetric neural network architecture including a first half of layers 110 and a second half of layers 120. The first half of layers 110 is configured to extract image features, reduce the feature map size, and retrieve the original image resolution, and the second half of the layers 120 is configured to identify the likely regions of interest (ROIs). In the very last layer 125, a two-channel map 130 is produced. The map 130 shows the probability of each pixel in the original image volume. The probability is either 0 (also referred to as the background class) or 1 (also referred to as the foreground class). Throughout this application, the CNN 100 is also referred to as a D-Net.

As illustrated in FIG. 1, the first half 110 of the CNN 100 includes a compression path, and the second half 120 of the CNN 100 incudes a decompression path to decompress the signal until the original size of the input signal is reached. In some embodiments, the input signal includes 3D images, such as OCT images.

In the CNN 100, convolutions are all applied with appropriate padding (i.e., adding extra pixels outside the original image). The first half 110 (i.e. left side) of the CNN 100 is divided into different stages that operate at different resolutions. In some embodiments, the first half 110 of the CNN 100 includes four stages 112a, 112b, 112c, and 112d as illustrated in FIG. 1. In some other embodiments, any other number of stages can also be used (e.g., anywhere from 2 stages to 16 stages). Each stage (112a through 112d) further includes one to three convolutional layers.

In addition, each stage (112a through 112d) is configured such that the stage learns a residual function. More specifically, the input of each stage (112a through 112d) is used in the convolutional layers and processed through the non-linearity. The input of each stage (112a through 112d) is also added to the output of the last convolutional layer of that stage in order to enable learning a residual function. More specifically, a residual connection (also referred to as a “skip connection”) data is set aside at an early stage and then added to the output later downstream (e.g., by adding together two matrices). These skip connections, or residual connections, can recover some of the information that is lost during down sampling. In other words, some of the raw data is persevered and then added later in the process. This approach can be beneficial for convergence of deep networks. More information about this approach can be found in Drozdzal, Michal, et al. “The importance of skip connections in biomedical image segmentation,” Deep Learning and Data Labeling for Medical Applications. Springer, Cham, 2016. 179-187, which is incorporated herein in its entirety. This architecture ensures convergence in a fraction of the time that would otherwise be used by a similar network that does not learn residual functions.

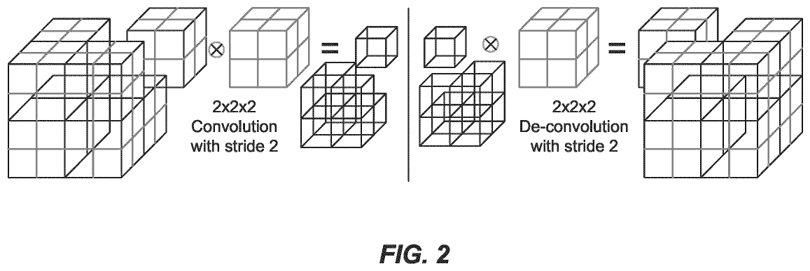

The convolutions performed in each stage use volumetric kernels (also referred to as filters). In some embodiments, the dimensions of the volumetric kernels can include 5×5×5 voxels. In some embodiments, any other dimensions can also be used. As the data proceeds through different stages 112a to 112d along the compression path in the first half 110 of the CNN 100, the resolution of the data is reduced at each stage (112a through 112d). This reduction of resolution is performed through convolution with 2×2×2 voxels wide kernels applied with stride 2 (see, e.g., FIG. 2 below). Since the second operation extracts features by considering only non-overlapping 2×2×2 volume patches, the size of the resulting feature maps is halved. This strategy can serve a similar purpose as pooling layers, which are usually inserted in-between successive convolution layers to progressively reduce the spatial size of the representation to reduce the amount of parameters and computation in a network.

Replacing pooling operations with convolutional operations in the CNN 100 can reduce the memory footprint during training for at least two reasons. First, the CNN 100 can operate without any switches to map the output of pooling layers back to their inputs that are otherwise used in conventional back-propagation. Second, the data can be better understood and analyzed by applying only de-convolutions instead of un-pooling operations.

Moreover, since the number of feature channels doubles at each stage 112a through 112d of the first half 110 (i.e. compression path) of the CNN 100, and due to the formulation of the model as a residual network, these convolution operations can be used to double the number of feature maps as the resolution of the data is reduced. In some embodiments, parametric Rectified Linear Unit (PReLU) nonlinearities are applied in the CNN 100. In some embodiments, leaky ReLU (LReLU) can be applied in the CNN 100. In some embodiments, randomized ReLU (RReLU) can be applied in the CNN 100.

Downsampling further allows the CNN 100 to reduce the size of the signal presented as input and to increase the receptive field of the features being computed in subsequent network layers. Each of the stages 112a to 112d in the first half 110 of the CNN 100 computes a number of features that is two times higher than the one of the previous layer.

The second half 120 of the CNN 100 extracts features and expands the spatial support of the lower resolution feature maps in order to gather and assemble information to output the two channel volumetric segmentation 130. The two features maps computed by the very last convolutional layer 125, having 1×1×1 kernel size and producing outputs of the same size as the input volume, are converted to probabilistic segmentations of the foreground and background regions by applying soft-max voxelwise.

The second half 120 of the CNN 100 also includes several stages 122a to 122d. After each stage 122a through 122d, a de-convolution operation is employed in order increase the size of the inputs (see, FIG. 2 below) followed by one to three convolutional layers involving half the number of 5×5×5 kernels employed in the previous layer. Similar to the first half 110 of the CNN 100, residual functions are employed in the convolutional stages 122a to 122d.

In the CNN 100, the features extracted from early stages in the first half 110 are forwarded to the second half 120, as illustrated by horizontal connections in FIG. 1. This architecture can gather fine grained detail that would be otherwise lost in the compression path and accordingly improve the quality of the final contour prediction. These connections can also improve the convergence time of the model.

FIG. 2 illustrates the decrease and increase of data size in the CNN 100 shown in FIG. 1 via convolution and de-convolutions, respectively, according to embodiments. In the compression path (i.e., first half 110 in the CNN 100), convolutions with appropriate stride can be used to reduce the size of the data. Conversely, de-convolutions in the second half 120 can increase the data size by projecting each input voxel to a bigger region through the kernel.

The CNN 100 can be trained end-to-end on a dataset of medical images, such as OCT images. In some embodiments, the training images include 3D images (also referred to as volumes). The dimensions of the training images can include, for example, 128×128×64 voxels, although any other dimensions can also be used. The spatial resolution of the images can be, for example, about 1×1×1.5 millimeters.

The training of the CNN 100 can include augmenting the original training dataset in order to obtain robustness and increased precision on the test dataset. In some embodiments, during every training iteration, the input of the CNN 100 can include randomly deformed versions of the training images. These deformed training images can be created using a dense deformation field obtained through a 2×2×2 grid of control-points and B-spline interpolation. In some embodiments, this augmentation can be performed “on-the-fly”, prior to each optimization iteration to alleviate the otherwise excessive storage demand.

In some embodiments, the intensity distribution of the data can be varied. For example, the variation can be created by adapting, using histogram matching, the intensity distributions of the training volumes used in each iteration, to the intensity distribution of other randomly chosen scans belonging to the dataset.

More information about the CNN architecture can be found in Milletari F, Navab N, and Ahmadi S-A. V-net: Fully convolutional neural networks for volumetric medical image segmentation. In: 3D Vision (3DV), 2016 Fourth International Conference on. IEEE; 2016. p. 565-571, which is incorporated herein in its entirety.

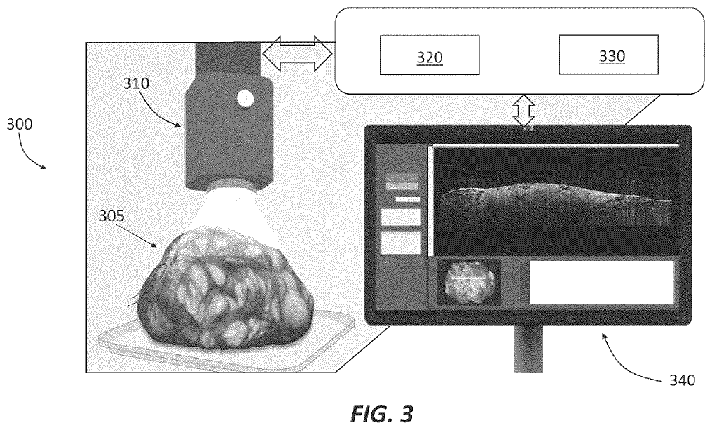

FIG. 3 illustrates a schematic of a system 300 for processing medical images using a CNN that is described herein (e.g., the CNN 100 shown in FIG. 1). The system 300 includes an imager 310 configured to acquire at least one image of a tissue 305 (e.g., a tissue sample image), a memory 320 operably coupled to the imager 310 and configured to store processor executable instructions, and a processor 330 operably coupled to the imager 310 and the memory 320. Upon execution of the processor executable instructions, the processor 330 is configured to train a CNN using sample images (e.g., training images) and implement the CNN to identify at least one ductal carcinoma in situ (DCIS) in the image acquired by the imager 310.

Suitable examples of imagers include imaging devices and systems disclosed in U.S. patent application Ser. No. 16/171,980, filed Oct. 26, 2018, published as U.S. Patent Application Publication No. 2019/0062681 on Feb. 28, 2019, and U.S. patent application Ser. No. 16/430,675, filed Jun. 4, 2019, the disclosures of each of which are incorporated herein by reference.

The CNN can be substantially similar to the CNN 100 shown in FIG. 1 and described above. In some embodiments, the sample images used for training the CNN can also be acquired by the imager 310. In some embodiments, the sample images used for training the CNN can be acquired by another imager and can be pre-processed (e.g., augmentation).

In some embodiments, the imager 310 can be configured to acquire OCT images. In some embodiments, the imager 310 can be placed in an operation room to acquire OCT images of the tissue 305 that is excised from a patient, and the system 300 is configured to determine a probability of abnormality associated with the OCT images and to generate annotated images to advise the surgeon whether additional excision is needed. In some embodiments, the system 300 is configured to generate the annotated image within 30 seconds (e.g., about 30 seconds, about 25 seconds, about 20 seconds, about 10 seconds, about 5 seconds, or less, including any values and sub ranges in between). In some embodiments, the annotated image can include an indication (e.g., highlighting, marking, etc.) of a probability of abnormality of the ROI. For example, different highlighting can be used to indicate whether different ROIs are benign or malignant.

In some embodiments, the system 300 can further include a display 340 operably coupled to the processor 330 and the memory 320. The display 340 can be configured to display the annotated image generated by the CNN. The annotated image can highlight regions of interest (e.g., regions including DCIS). In some embodiments, the annotated image includes a map of binary probabilities, i.e., each pixel either has a probability of 0 or 1, indicative of a probability of abnormality. In some embodiments, each pixel in the annotated image can have a possible pixel value between 0 and an integer number N, where N can be 2, 4, 8, 16, 32, or higher. In some embodiments, the annotated image is in grey scale. In some embodiments, the annotated image can be a color image. For example, the annotated image can use red color to highlight regions of interests. In some embodiments, the pixel values can be presented in a heatmap output, where each voxel can have a probability of abnormality between 0 and 1. For example, more red can mean higher probability of abnormality. In some embodiments, the annotated image can include an identifier (e.g., a pin, a flag, etc.) to further highlight regions of interested.

In some embodiments, the display 340 displays the annotated image and a surgeon then makes decision about whether to perform additional excision based on the annotated image. In some embodiments, the system 300 is further configured to calculate a confidence level about the necessity of excising additional tissues.

In some embodiments, the processor 330 can include a graphic processing unit (GPU). The capacity of the random access memory (RAM) in the processor 330 can be substantially equal to or greater than 8 Gb (e.g., 8 Gb, 12 Gb, 16 Gb, or greater, including any values and sub ranges in between). In some embodiments, the processor 330 can include an Nvidia GTX 1080 GPU having 8 Gb memory. In these embodiments, the system 300 may take about 15 seconds to produce DCIS labels for input volumes having a dimension of about 128×1000×500. In some embodiments, the processor 330 includes one GPU card, 2 CPU cores, and 16 Gb of available RAM.

In operation, a training process is performed to configure the setting of the CNN in the system 300 such that the CNN can take unknown images as input and produce annotated images with identification of regions of interest. In general, during the training process, a set of sample images with expected results are fed into the CNN in the system 300. The expected results can be obtained via, for example, manual processing by experienced clinicians. The system 300 produces tentative results of the input images. A comparison is then performed between the tentative results and the expected results. The setting of the CNN (e.g., weights of each neuron in the CNN) is them adjusted based on the comparison. In some embodiments, a backpropagation method is employed to configure the setting of each layer in the CNN.

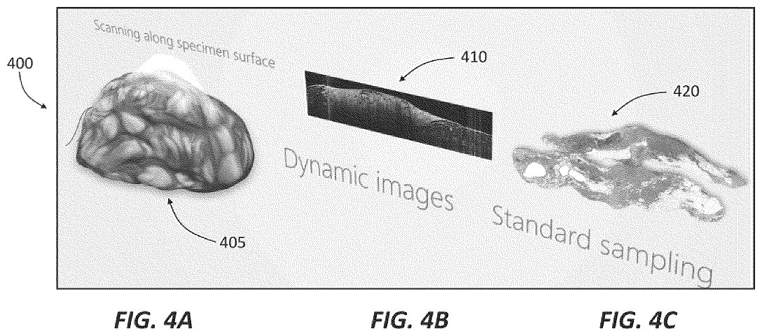

FIGS. 4A-4C illustrate a method 400 of obtaining the sample images with expected results (also referred to as ground truth) to train the CNN, according to an embodiment. In the method 400, an OCT scanning is performed on a tissue 405 as illustrated in FIG. 4A. The scanning generates OCT images 410 as shown in FIG. 4B. The tissue 405 is then physically cut and sectioned to obtain permanent section histology 420 for the corresponding area scanned by OCT, as illustrated in FIG. 4C. Readers (e.g., clinicians) can then navigate through the OCT images 410 to find correlation to any given permanent section slide in the histology 420. Once two areas (i.e. one in the OCT date 410 and one in the histology 420) are correlated, the regions of disease identified by readers can be identified in the OCT data as the expected results for training the CNN.

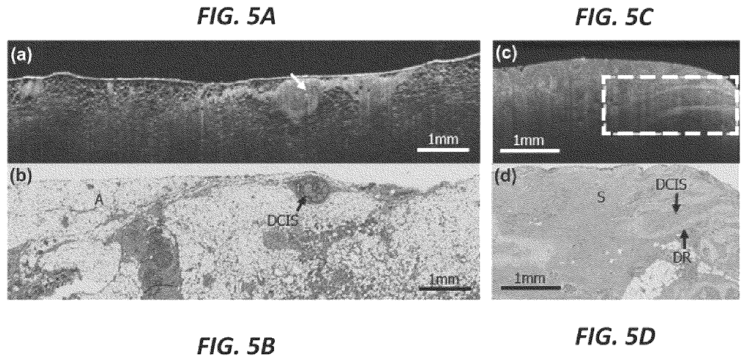

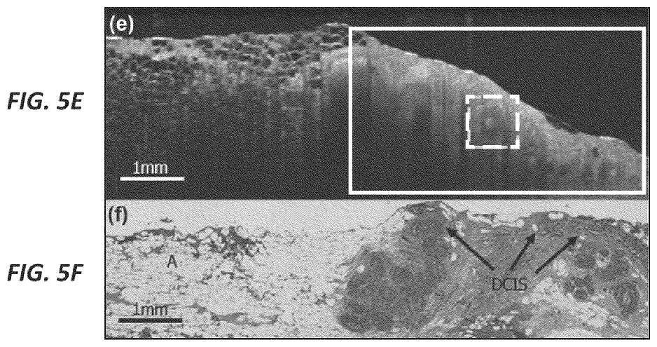

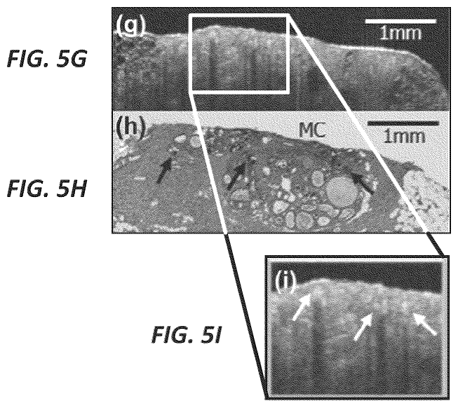

FIGS. 5A-5I show representative optical coherence tomography (OCT) B-scans and corresponding permanent section histology of DCIS cases. FIG. 5A shows a first sample OCT image (e.g., training image) and FIG. 5B shows a first histology corresponding to the OCT image shown in FIG. 5A. A DCIS is identified in the histology shown in FIG. 5B, and the corresponding region in the OCT image is accordingly identified (by a white arrow in FIG. 5A).

FIG. 5C shows a second sample OCT image (e.g., training image) and FIG. 5D shows a second histology corresponding to the OCT image shown in FIG. 5C. In this set of images, the DCIS has a tubular shape, compared to the solid shape in FIGS. 5A and 5B. FIG. 5E shows a third sample OCT image (e.g., training image) and FIG. 5F shows a third histology corresponding to the OCT image shown in FIG. 5E. In this set of images, three regions of DCIS are identified (see, FIG. 5F). FIG. 5G shows a fourth sample OCT image (e.g., training image), FIG. 5I shows a magnified region of the OCT image in FIG. 5G, and FIG. 5H shows a fourth histology corresponding to the OCT image in FIG. 5G. These correlations (i.e. between FIGS. 5A and 5B, between FIGS. 5C and 5D, between FIGS. 5E and 5F, and between FIGS. 5G and 5H) provide the ground truth for training the CNN in the system 300, which can be used to assign or associate ground-truth labels with the sample OCT images. In an embodiment, the ground-truth labels can be indicative of an abnormality of regions of tissue depicted in the set of training images (e.g., a likelihood or probability of abnormality of the regions of tissue). In an embodiment, the ground-truth labels can be voxel-wise labels.

To train the CNN in the system 300, a number of input volumes, and their corresponding voxel-wise labels are used. The goal of training is to find the weights of different neurons in the CNN, such that the CNN can predict the labels of the input images as accurately as possible. In some embodiments, a specialized optimization process called stochastic gradient descent (SGD, or one of its advanced variants) is used to iteratively alter the weights. In the beginning of training, the weights of neurons in the CNN are initialized randomly using a specific scheme. During each iteration, the CNN predicts a set of labels for the input volumes through the forward pass, calculates the error of label prediction by comparing against the ground-truth labels, and adjusts the weights accordingly through the backward pass. In this approach, backpropagation is used in the backward pass for calculation of gradients with respect to the error function. The value of this error function is obtained right after the forward pass based on ground-truth labels. By continuing this iterative process for multiple times (e.g., several tens of thousands of times), the weights of neurons in the CNN become well adjusted for the task of predicting labels of specific structure (or regions of interest, i.e. ROI) in any input volumes.

Due to the limited number of volume and label pairs available, various data augmentation techniques can be applied to increase the training efficacy. Neural networks are extremely capable of “memorizing” the input data in a very hard-coded fashion. In order to avoid this phenomenon and to enable proper “learning” of input representations, one can slightly manipulate the input data such that the network is encouraged to learn more abstract features rather than exact voxel location and values. Data augmentation refers to techniques that can tweak the input volumes (and their labels) in order to prepare a wider variety of training data.

In general, the input volumes and their corresponding labels can be manipulated such that they can be taken as new pairs of data items by the CNN. In some embodiments, the manipulation can include translation, i.e., 3D rigid movement of the volume in the space. In some embodiments, the manipulation can include horizontal flip, i.e., mirroring the images. In some embodiments, the manipulation can include free-form deformation, i.e., deforming the structures present in the volumes to a random form.

In some embodiments, prior to DCIS detection, the CNN in the system 300 can be trained using benign ductal structures and adipose tissue (i.e. fat tissue). The segmentation results of the mentioned tissue types are shown to be extremely accurate upon visual inspection, even for completely unseen OCT volumes. The results also demonstrate the capability of the CNN in the system in learning virtually any well-defined ROI given enough data.

One challenge in using the CNN in the system 300 for automatic tissue segmentation in OCT is defining the proper ROIs. The strength of the CNN is that the CNN can be trained to produce label maps for virtually any ROI as long as the ROI definition has/includes an ordered structure. In addition, depending on the complexity of the ROI type (e.g., in terms of the variety of image/visual features), different orders of sample sizes may be needed. In some embodiments, the label maps can be maps of the binary probability of abnormality (e.g., 0 or 1) of each pixel of a ROI in an image. The binary probability can represent, for example, whether each pixel has a low or high likelihood of abnormality.

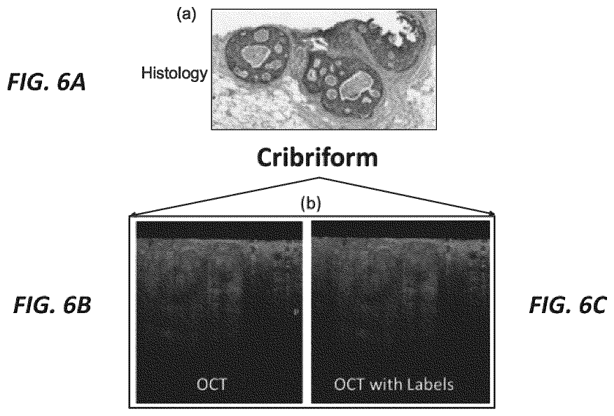

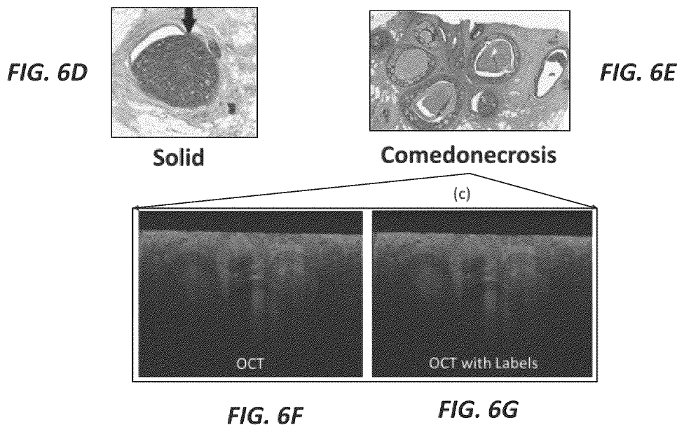

FIGS. 6A-6G show an example labeling of DCIS for training and using the system 300, according to an embodiments. FIG. 6A shows histology images including cribriform and solid types of DCIS, respectively. FIG. 6B shows an OCT image corresponding to the histology in FIG. 6A and FIG. 6C shows the same OCT image in which the DCIS region is labeled. These types of DCIS usually do not have entirely distinct features. In some embodiments, these two types of DCIS are labelled only if they show similarities to comedo type (i.e., a duct with some debris or calcifications or necrosis present, as shown in FIG. 6D).

FIG. 6E shows a histology including a comedonecrosis type of DCIS. FIG. 6F shows an OCT image corresponding to the histology in FIG. 6E, and FIG. 6G shows the same OCT image with labeling of the DCIS region. The comedo type has a donut-shape structure that is consistently present in a number of consecutive B-scans.

In some embodiments, the labelling can be performed on structures such as necrotic core, large calcification, or groups of calcifications. These structures tend to clearly indicate that the corresponding duct is either suspicious or benign, thereby facilitating subsequent review by a reader.

At least two strategies can be employed to perform the labeling. In some embodiments, the input data has a high resolution (e.g., about 15 μm or finer), in which case the labeling can be performed on a subset of the input images. For example, the labeling can be performed on one out of every N B-scans, where N is a positive integer. In some embodiments, N can be about 5 or greater (e.g., about 5, about 10, about 15, about 20, or greater, including any values and sub ranges in between), depending on the resolution of the input data. In some embodiments, the input data has a standard resolution (e.g., about 100 μm). In these embodiments, the labeling can be performed on all consecutive B-scans.

In operation, the accuracy of the labeling by the CNN in the system 300 can depend on the quality of the volumetric images. In general, a more isotropic image can lead to a more accurate prediction of labels. Therefore, it can be helpful to optimize the scanning protocols so as to obtain closer to isotropic voxel resolutions under the same time constraints. In some embodiments, isotropic voxel resolutions can be obtained by performing the spatial sampling uniformly in all directions. In some embodiments, the data can be resampled such that the spatial sampling is uniform. For example, a series of high resolution slices of the specimen can be acquired, followed by resampling so as to obtain isotropic voxel resolutions.

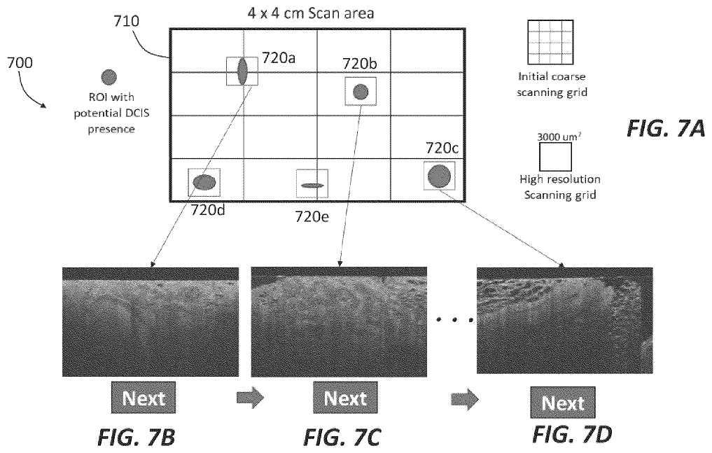

In addition, the reading of OCT images, even with the labeling by the CNN in the system 300, can still be a challenge due to the large image areas to be assessed. To address this challenge, FIGS. 7A-7D illustrate a method 700 of medical imaging processing using a tiered (or hierarchical) approach. In the method 700, a coarse scan is performed on a sample divided into a grid 710, as illustrated in FIG. 7A. For example, the coarse scan with isotropic resolution can be performed on a sample having a dimension of about 4 cm×4 cm. The coarse scan is employed to find suspect ROIs 720a to 720e using the predictions of the CNN in the system 300. In some embodiments, the pixel density of the coarse scan can depend on the time available in the operating room and the pre-op diagnosis (e.g., level of risks of observing infiltrating regions of malignancy). In general, a higher pixel density can increase the probability of finding all suspect ROIs.

Once the suspect ROIs 720a to 720e are detected, the method 700 proceeds to fine scan of each ROI. The fine scan is performed with a much higher resolution (e.g., about 20 μm to about 250 μm). For example, each ROI can have an area of about 5000 μm² or less (e.g., about 5000 μm², about 4000 μm², about 3000 μm², about 2000 μm², about 1000 μm², or less, including any values and sub ranges in between). The CNN is also employed to assess each ROI to detect DCIS.

In some embodiments, the reading process can be automated with the user clicking a “next” button to review the findings of the system 300. For example, as the user clicks the next button, the system 300 can sequentially display the annotated images of the regions 720a, 720b, and 720c, illustrated in FIGS. 7B, 7C, and 7D, respectively. During this process all the user would need to do is to click a few buttons to select scan area and start the automated process all the way to the reading phase, which can also be substantially automated with ‘next’ buttons.

While various inventive implementations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive implementations described herein. More generally, those skilled in the art will readily appreciate that all parameters and configurations described herein are meant to be exemplary inventive features and that other equivalents to the specific inventive implementations described herein may be realized. It is, therefore, to be understood that the foregoing implementations are presented by way of example and that, within the scope of the appended claims and equivalents thereto, inventive implementations may be practiced otherwise than as specifically described and claimed. Inventive implementations of the present disclosure are directed to each individual feature, system, article, and/or method described herein. In addition, any combination of two or more such features, systems, articles, and/or methods, if such features, systems, articles, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one implementation, to A only (optionally including elements other than B); in another implementation, to B only (optionally including elements other than A); in yet another implementation, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one implementation, to at least one, optionally including more than one, A, with no B present (and optionally including elements other than B); in another implementation, to at least one, optionally including more than one, B, with no A present (and optionally including elements other than A); in yet another implementation, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system, comprising:
an imager configured to image tissue;
a memory; and
a processor operatively coupled to the imager and memory, the processor configured
train a convolutional neural network (CNN) in an iterative process using a set of training images and a set of histology images, the set of training images associated with a set of ground-truth labels, the set of ground-truth labels indicative of an abnormality of regions of tissue depicted in the set of training images, and each histology image uniquely correlated with a training image from the set of training images and depicting the region of tissue of that training image;
receive a tissue sample image from the imager, the tissue sample image including a region of interest (ROI); and
determine, using the CNN after the training, a probability of abnormality of the ROI.

2. The system of claim 1, wherein the imager includes an optical coherence tomography (OCT) device.

3. The system of claim 1, wherein the tissue sample image is a three-dimensional (3D) image formed from two-dimensional scans of a tissue sample.

4. The system of claim 1, wherein the processor is configured to train the CNN by:
generating, using the CNN implementing a set of weights, a set of predicted labels for the set of training images;
comparing the set of predicted labels to the set of ground-truth labels to define an error function; and
adjusting the set of weights in one or more iterations of the generating and the comparing to reduce a value of the error function.

5. The system of claim 1, wherein the processor is configured to train the CNN by using stochastic gradient descent (SGD) in the iterative process to adjust a set of weights implemented by the CNN.

6. The system of claim 1, wherein the set of ground-truth labels are based on permanent section histology of regions of tissue imaged in the set of training images.

7. The system of claim 1, wherein the processor is further configured to: manipulate the set of training images using a set of data augmentation techniques to produce an augmented set of training images,
the processor configured to train the CNN using the set of training images and the augmented set of training images.

8. The system of claim 7, wherein the set of data augmentation techniques includes at least one of: a translation of a set of structures present in the set of training images, a deformation of the set of structures, or a mirroring of a subset of the training images.

9. The system of claim 1, wherein the processor is further configured to: generate an annotated image of the tissue sample image, the annotated image including an indication of the probability of abnormality of the ROI.

10. The system of claim 9, wherein the annotated image represents a map of binary probabilities of abnormality of each pixel of the tissue sample image.

11. The system of claim 1, further comprising:
a display configured to display the annotated image with highlighting to reflect the indication of the probability of abnormality of the ROI,
the processor operatively coupled to the display.

12. The system of claim 1, wherein the ROI is associated with a ductal carcinoma in situ (DCIS).

13. An apparatus, comprising:
a memory; and
a processor operatively coupled to the memory, the processor configured to:
receive a set of training images, each training image from the set of training images depicting a region of tissue;
receive a set of histology images, each histology image uniquely correlated with a training image from the set of training images and depicting the region of tissue of that training image;
train a convolutional neural network (CNN) in an iterative process using the set of training images and a set of labels applied to structures depicted in the set of training images based on the set of histology images;
receive a tissue sample image from an imager, the tissue sample image including a ROI; and
determine, using the CNN after the training, a probability of abnormality of the ROI.

14. The apparatus of claim 13, wherein the processor is further configured to:
receive a coarse scan of the tissue sample from the imager;
detect, using the CNN after the training, the ROI in the coarse scan; and
in response to the detecting, cause the imager to fine scan the ROI, the tissue sample image including a fine scan of the ROI.

15. The apparatus of claim 14, wherein the coarse scan and the fine scan have isotropic resolution.

16. The apparatus of claim 14, wherein the fine scan has a resolution of about 20 μm to about 250 μm.

17. The apparatus of claim 13, wherein the processor is configured to train the CNN by:
generating, using the CNN implementing a set of weights, a set of predicted labels for the structures;
comparing the set of predicted labels to the set of labels applied to the structures to define an error function; and
adjusting the set of weights of the CNN in one or more iterations of the generating and the comparing to reduce a value of the error function.

18. The apparatus of claim 13, wherein the processor IS further configured to:
manipulate the set of training images using a set of data augmentation techniques to produce an augmented set of training images,
the processor configured to train the CNN using the set of training images and the augmented set of training images.

19. The apparatus of claim 13, wherein the processor IS further configured to:
generate an annotated image of the tissue sample image, the annotated image including an indication of the probability of abnormality of the ROI.

20. A method, comprising:
receiving a set of training images, each training image from the set of training images depicting a region of tissue;
receiving a set of histology images, each histology image uniquely correlated with a training image from the set of training images and depicting the region of tissue of that training image;
training a convolutional neural network (CNN) in an iterative process using the set of training images and a set of labels applied to structures depicted in the set of training images based on the set of histology images;
receiving a tissue sample image from an imager, the tissue sample image including a region of interest (ROI); and
determining, using the CNN after the training, a probability of abnormality of the ROI.
